(19) United States
(12) Patent Application Publication
(10) Pub. No.: US 2002/0006437 A1
(43) Pub. Date: Jan. 17, 2002

(54) Non-migration tissue capsule

(76) Inventors: Jamie M. Grooms, Alachua, FL (US); Tom Sander, Alachua, FL (US)

Correspondence Address: Timothy H. Van Dyke, Bencen & Van Dyke, P.A., 1630 Hillcrest Street, Orlando, FL, 32803, US

(21) Appl. No.: 09/847,939

(22) Filed: May 2, 2001

Related U.S. Application Data
(60) Provisional application No. 60/201,253, filed on May 1, 2000.

(51) Int. Cl. A61K 9/48; A61K 9/64

(52) U.S. Cl. 424/451; 424/456

(57) ABSTRACT

Disclosed herein are novel non-migration tissue capsules and methods of using the same. The capsules are non-allergenic and biocompatible and can be implanted into a patient in need thereof to correct any number of defects and abnormalites whether congenital or brought about by aging, surgery, and injury. Preferably, medically useful substances can be disposed on or within the subject tissue capsules whereby the medically useful substances are protected and retained at the site of need by the capsule.

### CROSS-REFERENCE TO RELATED APPLICATIONS

The subject application claims the benefit under 35 USC §119 of Provisional Application No. 60/201,253 filed May 1, 2000.

### BACKGROUND OF THE INVENTION

In the field of orthopedic implants, it is desirous to provide an implant that is provided with an osteogenic material or some other medically useful substance. However, a common problem with many implants containing osteogenic material is that this material is squeezed out of the implant, or otherwise migrates away from the intended target area. Accordingly, a need exists in the art for a means to implant osteogenic and other materials into a patient that is able to withstand internal physical forces and stresses to prevent the unwanted migration of these materials from the region of implantation.

### SUMMARY OF THE INVENTION

The subject invention relates to a biocompatible implant comprising an osteogenic and/or regenerative material covered by a protective capsule made from tissue or other biocompatible substance, thereby preventing the undesirable migration of the osteogenic and/or regenerative material from the site of implantation once positioned in the patient.

### DESCRIPTION OF THE DRAWINGS

FIG. 1 shows a perspective view of an arcuate embodiment of the subject invention that comprises pores therethrough.

FIG. 2 shows a perspective view of an elongated embodiment of the subject invention that comprises pores therethrough.

FIG. 3 shows a perspective view of another elongated embodiment of the subject invention that comprises elongated slits for pores.

FIG. 4 shows an additional embodiment of the subject invention that is configured as a rod having a plurality of perforations therethrough.

FIG. 5 shows a perspective view of an additional embodiment that is concaved for holding a medically useful substance.

FIG. 6 shows an alternative embodiment of the subject invention that is configured as an elongated hollow tube having an open end and a closed end, wherein medically useful substances can be disposed in the hollow space.

### DESCRIPTION OF THE PREFERRED EMBODIMENTS

As used herein, the term “biocompatible” refers to the characteristic of not causing an allergic or otherwise adverse reaction in a recipient. Those skilled in the art will appreciate, in view of the teachings herein, that the subject implant can have a myriad of shapes and sizes, including, but not limited to, cylindrical, rectangular, triangular, oblong-shaped, circular, trapezoidal, among many others.

The protective capsule of the subject implant can be made of any suitable material that is, preferably, biocompatible and possess a requisite amount of structural strength to prevent the undesirable migration of the osteogenic or, regenerative material from the site of need.

The choice of the capsule material will vary depending on the specific application in which the capsule is used. Physical and chemical characteristics such as, e.g., biocompatibility, biodegradability, strength, rigidity, interface properties, and even cosmetic appearance may be considered in choosing a capsule material. In applications where the capsule will bear high levels of stress and thereby require high levels of strength and rigidity, suitable materials would include, e.g., bone (cortical and/or cancellous); mineralized collagen (see U.S. Pat. No. 5,231,169); bioceramics such as hydroxyapatite, bioglass, aluminates, tricalciumphosplate, and calcium phosplate; polymeric materials such as acrylic ester polymers and lactic acid polymers (see U.S. Pat. Nos. 4,521,909, and 4,563,489).

In other embodiments of the subject implant where a lesser amount of stress bearing is needed, the capsule may be made of e.g., gelatin, collagen, demineralized bone matrix (DBM), collagen and glycosaminoglycan (GAG) (U.S. Pat. No. 4,505,266). See U.S. Pat. No. 5,904,718 for examples of gelatin capsules.

The type of medically useful substances contained in the capsule will vary according to intended application. In preferred embodiments, the medically useful substances include, but are not limited to, commercially available bone pastes such as those disclosed in WO98/40113, collagen and insoluble collagen derivatives; gelatin; hydroxyapatite, etc., and soluble solids and/or liquids dissolved therein, e.g., antiviricides, particularly those effective against viruses such as HIV and hepatitis; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digestors; antitumor agents; fibronectin; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; nucleic acids; bioerodable polymers such as those disclosed in U.S. Pat. Nos. 4,764,364 and 4,765,973, and combinations of any of the foregoing. The amounts of such medically useful substances can vary widely with optimum levels being readily determined in a specific case by routine experimentation.

According to a preferred aspect, medically useful substances used in accord with the teachings herein comprise one or more growth factors. The term “growth factor” as used herein refers to a polynucleotide molecule, polypeptide molecule, or other related chemical agent that is capable of effectuating differentiation of cells. Examples of growth factors as contemplated for use in accord with the teachings herein include an epidermal growth factor (EGF), transforming growth factor-alpha (TGF alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), and/or platelet derived growth factor (PDGF). Growth factors for use in accord with the teachings herein can be extracted from allograft, xenograft and/or autograft tissue, or can be produced by recombinant/genetic means, or be encoded by nucleic acids associated with appropriate transcriptional and translational elements.

According to a particular aspect, the subject invention is directed to a method comprising encapsulating one or more medically useful substances that comprise the ability to stimulate generation of specific tissues; and implanting said encapsulated one or more medically useful substances in an area of a patient in need of repair of specific tissues, or further generation of such tissues.

In an alternative embodiment, the subject biomedical capsule contains Platelet Rich Plasma (PRP), or growth factors isolated from PRP. PRP obtained from autograft blood has been shown to increase the rate of healing of autogenic grafts. Current methods of applying PRP to such grafts involves the removal of blood from a patient (plasmapheresis), centrifuging the blood, drawing off the PRP layer, and applying the PRP to the graft, which occurs just prior to surgery. There is a need in the art to alleviate the costs and inefficiencies involved with the current methods. Accordingly, in a further embodiment of the subject invention, provided is a method of obtaining an allograft and/or xenograft source of PRP for use in graft implantation. In a specific embodiment, the PRP is obtained by procuring blood from a cadaveric donor (such as by conventional exsanguination techniques) or procuring blood (preferably expired blood as to avoid depletion of blood earmarked for other purposes) from blood banks, and centrifuging the obtained blood to separate the PRP from other blood components via conventional methods. Preferably, PRP is obtained from a cadaveric donor. The isolation of PRP from sources other than autogenous (recipient) allows for the manipulation and use of the PRP well prior to surgery, whereby the inefficient removal and treatment of blood from the recipient is alleviated.

Furthermore, it is generally believed in the art that the beneficial effects of PRP are due to the presence of various growth factors, such as platelet derived growth factor (PDGF), platelet derived angiogenic growth factor (PDAF), platelet derived epidermal growth factor (PDEGF), and transforming growth factor (TGF-beta). Allogenic and/or xenogenic blood provides a vast and untapped source for PRP and growth factors. In a specific embodiment, platelets are isolated from allogenic and/or xenogenic sources as described above, and growth factors are partially purified or purified from these isolated platelets via conventional methods (see, e.g., U.S. Pat. Nos. 4,479,896; 4,861,757; or 4,975,526). As used herein, the term “partially purified” refers to a state of purification above that which is found in nature, or said differently, that is not achievable unless through manipulation by the hand of man. The term “purified” as used herein refers to a state of purification such that in a given sample comprising a given growth factor, the growth factor is 95% or greater, by weight, of the sample. Once they are partially purified or purified, the growth factors can be stored and/or distributed in a lyophilized or frozen form. Accordingly, the subject methods allow for the mass production of implants (autogenic, allogenic, and/or xenogenic) that have been treated with PRP, and/or growth factors isolated therefrom, that are readily usable in implantation surgeries, which also decreases the costs and inconvenience associated with conventional methods.

Preferably, PDGF used in this embodiment is obtained by the method disclosed in copending application U.S. Ser. No. 09/776,619, which is provided here in brief: (a) obtain outdated apheretically purified platelets (platelets present in 60-70 ml plasma); (b) keep platelets at 4° C.; (c) combine donor platelets into 500 ml centrifuge tubes; (d) centrifuge tubes at 8000×g 20 minutes at 4° C.; (e) remove plasma; (f) add 20 volumes of ice cold sterile saline to platelets and gently resuspend pellet; (this step is to remove as much plasma/serum components as possible); (g) re-centrifuge at 8000×g 20 min at 4° C. to repellet platelets; (h) to platelet pellet, add 10 volumes extraction buffer (1.45% Ethanol containing 150 ul concentrated HCl for every 50 ml of solution; or 2. 100 mM NaH₂PO₄; 1.5M NaCl; pH 7.4) and agitate overnight at 4° C. (12-16 hours); (i) pellet lysed platelet material by centrifugation at 12,000 rpm 20 minutes 4° C.; (j) remove platelet extract.

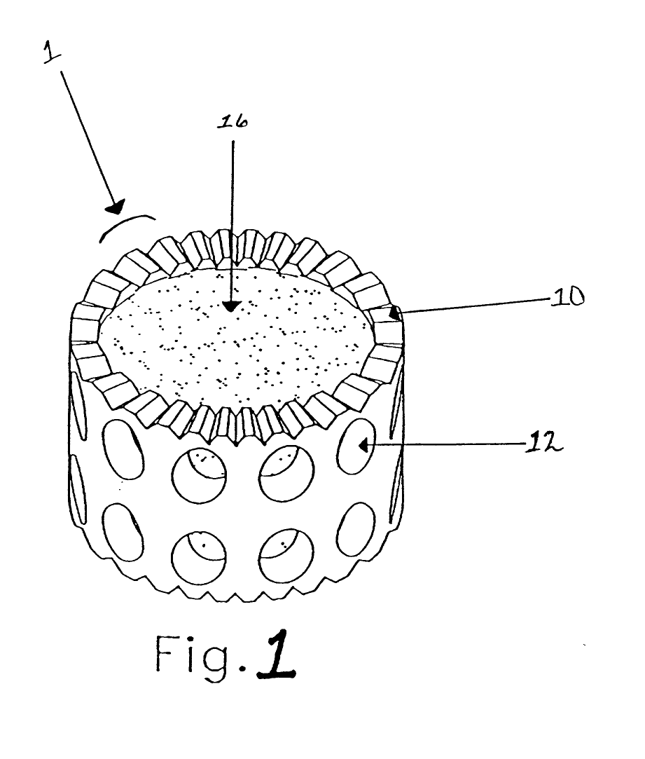

FIGS. 1-3 depict preferred embodiments of the subject invention. FIG. 1 shows an embodiment 1 of the invention that is specifically designed for implantation into the spine. The capsule 10 is preferably made of cortical or cancellous bone and defines a plurality of holes 12 therethrough which are in communication with the outersurface of the capsule 10. The holes can be provided in any suitable shape, such as circular, oblong, rectangular, etc. The number of holes provided will vary depending on the rigidity and strength required for a given use. The embodiments shown in FIG. 1-3 have encapsulated in the capsule 10 an osteogenic material 16. The osteogenic material is preferably bone paste as disclosed in WO99/38543 or other commercially available materials such as GRAFTON® (See U.S. Pat. No. 5,484,601), DYNAGRAFT® (See U.S. Pat. No. 5,707,962 and 5,786,327) or a collagen sponge loaded with one or more growth factors. While the preferred uses of the subject capsule are in the treatment of bone defects, injuries and/or wounds, an alternative use for the subject invention is for the implantation into the muscle of the patient to convert the muscle into bone.

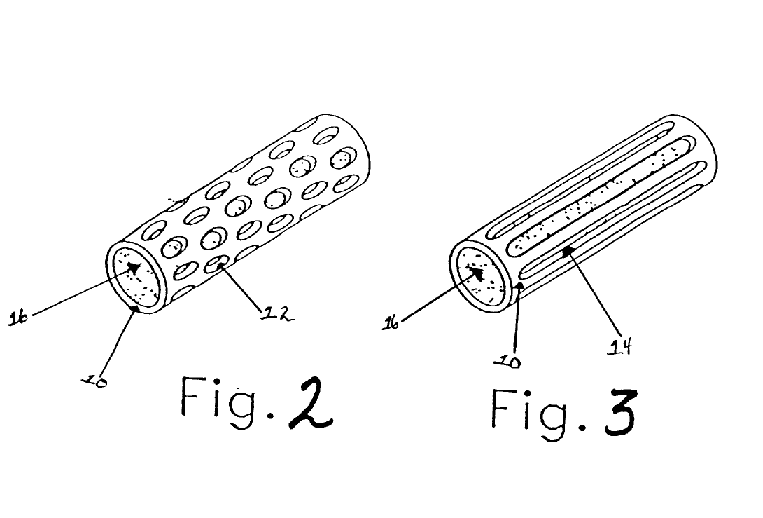

The embodiments shown in FIGS. 2 and 3 represent a more elongated, cylindrical, shape of the subject capsule. A preferred application for this embodiment is in the treatment of avascular necrosis of the hip, (such as by the methods disclosed in U.S. Pat. No. 5,755,809). FIG. 3 shows elongated holes 14 for allowing the osteogenic material to communicate with the outside of the capsule 10.

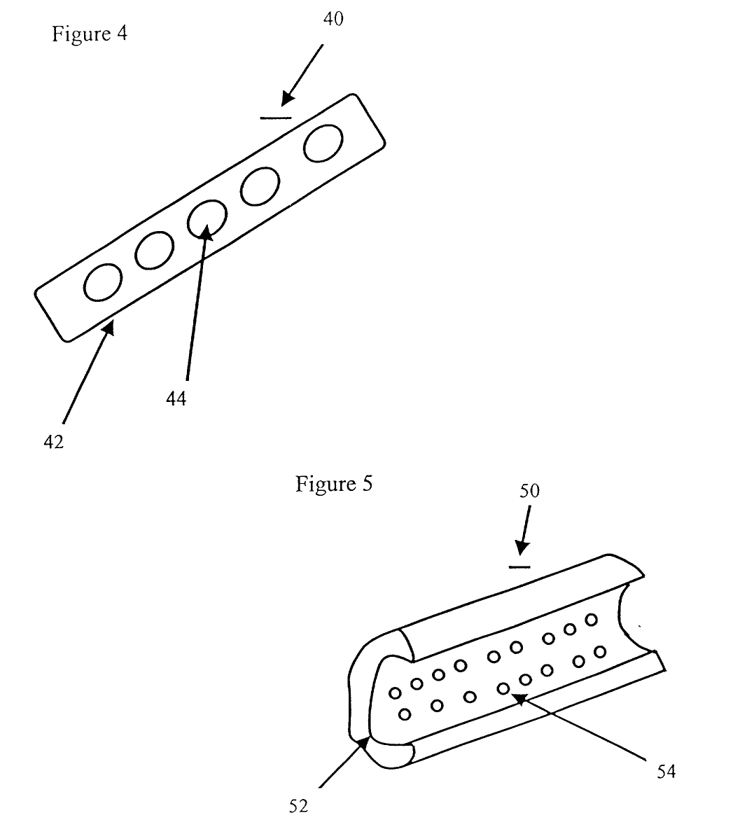

FIG. 4 shows an embodiment 40 of the subject invention whose preferred use is also in the treatment of avascular necrosis. In this embodiment, the capsule 42 is an elongated rod that has holes 44 or indentions for holding osteogenic material.

In an alternative embodiment, as shown in FIG. 5, the capsule 50 is comprised of a solid section of cortical or cancellous bone that has had one side bored out thereby forming a deep indention 52, or otherwise has an indention derived from the natural architecture of the bone, to hold osteogenic or regenerative material. Preferably, the capsule of this embodiment defines a plurality of holes 54 therethrough.

In a further embodiment, the subject capsule is directed to a section of cortical or cancellous bone which has had the center milled out thereby forming a channel through the section of bone. The center channel can be oriented along the longitudinal axis of the section of bone, or, alternatively, through a transverse axis of the section of bone. Preferably, the internal surface of the capsule that defines the channel has ridges or slots formed thereon which aid in the retention of osteogenic and/or regenerative, or other medically useful substances disposed within the channel.

In yet another embodiment, the subject biomedical implant is directed to a hollow gelatin capsule of any shape, but preferably oblong, wherein the gelatin capsule has an amount of a medically useful substance completely covered within its walls. As the gelatin begins to degrade after implantation, the medically useful substance is released over time. This embodiment may be especially useful to convert muscle tissue into bone.

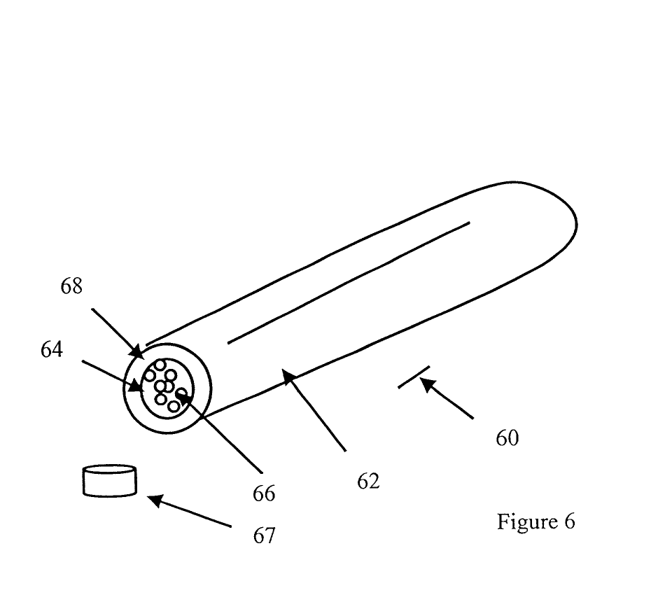

FIG. 6 shows an embodiment 60 of the subject invention that comprises an elongated capsule 62 that defines a center channel 64. Disposed within the channel is a medically useful substance 66. According to this embodiment, the elongated capsule completely encloses the center channel 64 except for an open end 68 at one of the two ends of the capsule. Preferably, a cap 67 can be secured by friction or otherwise to the open end. Even more preferably, the cap 67 is made of a degradable substance such as gelatin, which upon degradation allows the medically useful substance 66 to be released. A preferred medically useful substance for use with this embodiment is a collagen sponge infused with growth factors. Alternatively the capsule can define a plurality of holes therethrough.

The teachings of the references cited throughout the specification are incorporated herein by this reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A biomedical implant for implantation into a patient in need thereof, said biomedical implant comprising a capsule and a medically useful composition disposed within said capsule.

2. The biomedical implant of claim 1, wherein said capsule is made of tissue.

3. The biomedical implant of claim 1, wherein said capsule is made of bone, mineralized collagen, bioceramic material(s), polymeric material(s), gelatin, collagen, DBM, GAG, or combinations thereof.

4. The biomedical implant of claim 1, wherein said capsule is made of bone or gelatin.

5. The biomedical implant of claim 1, wherein said medically useful composition comprises collagen and insoluble collagen derivatives; gelatin; hydroxyapatite and soluble solids liquids dissolved therein; antiviricides; antimicrobials; amino acids; magainins; peptides; vitamins; inorganic elements; co-factors for protein synthesis; hormones; endocrine tissue or tissue fragments; synthesizers; collagenases; peptidases; oxidases; polymer cell scaffolds with parenchymal cells; surface cell antigen eliminators; angiogenic drugs optionally associated with polymeric carriers containing such drugs; collagen lattices; biocompatible surface active agents; antigenic agents; cytoskeletal agents; cartilage fragments; living cells; bioadhesives; growth factors; growth hormones; bone digestors; antitumor agents; fibronectin; cellular attractants; attachment agents; immuno-suppressants; permeation; nucleic acids; bioerodable polymers; and combinations of the foregoing.

6. The biomedical implant of claim 1, wherein said capsule defines a center channel wherein said medically useful substance is disposed.

7. The biomedical implant of claim 6, wherein said center channel has slots formed therein to aid in the retention of said medically useful composition disposed within said channel.

8. The biomedical implant of claim 1, wherein said biomedical implant is square, cylindrical, triangular, trapezoidal, oblong, or a combination thereof.

9. The biomedical implant of claim 1, wherein said capsule defines one or more holes therethrough.

10. The biomedical implant of claim 1, wherein said capsule is cylindrical in shape.

11. The biomedical implant of claim 10 comprising an outer shell of tissue configured to be an elongated, tube-like shape, wherein said outer shell comprises a closed end and an open end, and defines a hollow cavity;
wherein one or more medically useful substances are disposed in said hollow cavity.

12. The biomedical implant of claim 10 comprising an outer shell of tissue configured to be an elongated, tube-like shape, wherein said outer shell comprises a first open end and a second open end, and defines a hollow cavity;
wherein one or more medically useful substances are disposed in said hollow cavity.

13. The biomedical implant of claim 12, wherein said outer shell comprises a plurality of holes therethrough, whereby said one or more medically useful substances communicate with the outside of said outer shell.

14. The biomedical implant of claim 13 wherein said plurality of holes are elongated slits.

15. The biomedical implant of claim 1, wherein said implant comprises a segment of tissue configured as an elongated, rod, wherein said segment comprises a plurality of holes defined therethrough.

16. The biomedical implant of claim 1, wherein said implant comprises an outer shell of tissue configured to be a concave shape; and wherein said outer shell optionally comprises a plurality of holes therethrough.

17. A method for repairing damaged tissue, or otherwise generating further tissue, comprising the steps of encapsulating a medically useful composition by disposing said medically useful composition in a capsule thereby forming a loaded capsule; and implanting said loaded capsule into a patient in need thereof.

18. A method of stabilizing a femoral head comprising the steps of excising a section of bone from an anterolateral segment of said femoral head to thereby form a hole; and inserting into said hole a biomedical implant comprising a cylindrical capsule and a medically useful substance disposed within said capsule.

19. An article of manufacture comprising a biomedical implant for implantation into a patient in need thereof, said biomedical implant comprising a capsule and a medically useful composition disposed within said capsule; and a packaging container for enclosing said biomedical implant.

20. The article of manufacture of claim 13 wherein packaging container comprises a box, jar or bag, said packaging container being made of wood, paper, glass or plastics.

21. The article of manufacture of claim 13 further comprising a desiccant.
